(19) United States
(12) Patent Application Publication
(10) Pub. No.: US 2005/0273146 A1
(43) Pub. Date: Dec. 8, 2005

(54) Liquid perfluoropolymers and medical applications incorporating same

(75) Inventors: Joseph M. DeSimone, Chapel Hill, NC (US); Michael S. Williams, Santa Rosa, CA (US)

Correspondence Address: MYERS BIGEL SIBLEY & SAJOVEC, PO BOX 37428, RALEIGH, NC, 27627, US

(73) Assignee: SyneCor, LLC

(21) Appl. No.: 11/200,370

(22) Filed: Aug. 9, 2005

Related U.S. Application Data
(63) Continuation-in-part of application No. 11/020,779, filed on Dec. 22, 2004.
(60) Provisional application No. 60/532,853, filed on Dec. 24, 2003.
(60) Provisional application No. 60/535,765, filed on Jan. 12, 2004.

(51) Int. Cl. A61N 1/375; A61F 2/06; A61F 2/02; A61F 13/00; A61L 17/04; G02C 7/04

(52) U.S. Cl. 607/116; 623/1.44; 606/76; 606/231; 602/52; 623/1.42; 623/23.72; 351/160 R; 623/1.49; 128/898

(57) ABSTRACT

Liquid curable perfluoropolyether (PFPE) materials are provided for use as coatings, sealants, flexible fillers, and structural parts for a wide variety of medical applications, particularly where silicone has been utilized conventionally. The PFPE material is oxygen permeable and bacterial impermeable and may contain one or more pharmacological agents elutably trapped therewithin for delivery within the body of a subject.

### RELATED APPLICATION

This application is a continuation-in-part application of U.S. patent application Ser. No. 11/020,779, filed Dec. 22, 2004, which claims the benefit of U.S. Provisional Application No. 60/532,853 filed Dec. 24, 2003, and U.S. Provisional Application No. 60/535,765 filed Jan. 12, 2004, the disclosures of which are incorporated herein by reference in their entireties as if set forth fully herein.

### FIELD OF THE INVENTION

The present invention relates generally to polymers and, more particularly, to medical applications where polymers are utilized.

### BACKGROUND OF THE INVENTION

Many devices, such as surgical instruments, medical devices, prosthetic implants, contact lenses, and the like, are formed from polymeric materials. Polymeric materials conventionally utilized in the medical device industry for implantation within the bodies of subjects include, but are not limited to polyurethanes, polyolefins (e.g., polyethylene and polypropylene), poly(meth)acrylates, polyesters (e.g., polyethyleneterephthalate), polyamides, polyvinyl resins, silicone resins (e.g., silicone rubbers and polysiloxanes), polycarbonates, polyfluorocarbon resins, synthetic resins, polystyrene, and various bioerodible materials.

Silicone is characterized by high lubricity and thermal stability, extreme water repellence and physiological inertness. Accordingly, silicone has been widely used in the medical field in various applications such as adhesives, lubricants, surgical implants and prosthetics. Unfortunately, silicone may swell and/or shrink, particularly when contact occurs with solvents, for example, organic solvents. In addition, the surface energy of silicone may not be as low as desirable for certain applications where higher lubricity is necessary.

Accordingly, a need exists for improved polymeric materials for various medical applications, particularly applications where devices are implanted and inserted within the body of a subject.

### SUMMARY OF THE INVENTION

In view of the above discussion, liquid curable perfluoropolyether (PFPE) materials are provided for use as coatings, sealants, flexible fillers, and structural parts for a wide variety of medical applications, particularly where silicone has been utilized conventionally. PFPE materials utilized in accordance with embodiments of the present invention does not swell or shrink when contact occurs with solvents, including organic solvents. In addition, the surface energy of PFPE material is very low which allows PFPE material to be utilized for certain applications where high lubricity is necessary. Moreover, PFPE material is oxygen permeable and bacterial impermeable.

According to embodiments of the present invention, a method of repairing damage to skeletal portions of the body of a subject in situ, according to embodiments of the present invention, includes positioning an enclosure adjacent a damaged skeletal portion of a subject, injecting a liquid PFPE material into the enclosure, and curing the liquid PFPE material to form a structure that provides support to the skeletal portion. The liquid PFPE material may cure to a rigid state, a flexible state, or portions of the PFPE material may cure to respective rigid and flexible states. Exemplary skeletal damage that may be repaired according to embodiments of the present invention includes bone cracks, damaged vertebral bodies, damaged wear surfaces of joints, and damaged joints including, but not limited to, hips, knees, ankles, phalange joints, elbows, and wrists. One or more pharmacological agents may be elutably trapped within the cured PFPE material (or otherwise attached to the PFPE material), according to embodiments of the present invention. In addition, unwanted material, such as damaged material of a skeletal portion of a subject may be removed prior to positioning an enclosure and injecting PFPE material into the enclosure.

According to embodiments of the present invention, orthopedic devices are provided that are configured to be implanted within the body of a subject and that include an outer surface of oxygen permeable, bacterial impermeable PFPE material. Utilizing PFPE material with removable implants of any type is advantageous because tissue in-growth can be minimized, thus making removal of the implant safer and less traumatic.

According to embodiments of the present invention, orthopedic devices are provided that are configured to be implanted within the body of a subject and that include layers of uniaxially and biaxially oriented materials.

According to embodiments of the present invention, prosthetic devices deployed within the body of a subject may be repaired in situ using PFPE material. For example, damaged or unwanted material (e.g., a damaged surface portion) from a prosthetics device is removed, an enclosure is positioned at the location of the removed material, and a liquid PFPE material is injected into the enclosure. The PFPE material is then cured and the cured PFPE material serves as a replacement for or repair of prosthetics device material.

According to embodiments of the present invention, bandages and other wound healing devices (e.g., sutures) are provided that include oxygen permeable, bacterial impermeable PFPE material. Such wound healing bandages and devices may include one or more pharmacological agents for treating damaged tissue.

According to embodiments of the present invention, a method of applying a bandage to a portion of a body of a subject includes applying (e.g., spraying, swabbing, etc.) an oxygen permeable, bacterial impermeable liquid PFPE material onto a portion of the body of a subject, and then curing the liquid PFPE material such that the PFPE material forms a protective bandage that facilitates healing of underlying tissue.

According to embodiments of the present invention, artificial blood vessels are provided for insertion within the body of a subject and include oxygen permeable, bacterial impermeable PFPE material. One or more pharmacological agents may be elutably trapped within the PFPE material (or otherwise attached to the PFPE material).

According to embodiments of the present invention, a method of replacing in situ a portion of a blood vessel within the body of a subject includes injecting an oxygen permeable, bacterial impermeable liquid PFPE material into the lumen of a portion of an existing blood vessel to form an artificial blood vessel, and then curing the liquid PFPE material to produce a replacement for the blood vessel portion. The existing blood vessel serves as a mold for the liquid PFPE material. The replaced portion of the existing blood vessel may then be removed. If the lumen of the existing blood vessel portion is occluded or partially occluded, the occlusion may be removed prior to injection of the PFPE material.

According to embodiments of the present invention, intraluminal prostheses (e.g., stents) having tubular body portions that include oxygen permeable, bacterial impermeable PFPE material are provided. According to embodiments of the present invention, one or more pharmacological agents may be elutably trapped within the PFPE material (or otherwise attached to the PFPE material) of such an intraluminal prosthesis. The PFPE material may be configured to allow the one or more pharmacological agents to elute therefrom (e.g., at a predetermined rate) when an intraluminal prosthesis is deployed within a body of a subject. According to embodiments of the present invention, a pharmacological agent may be homogeneously distributed on the tubular body portion of an intraluminal prosthesis. Alternatively, a pharmacological agent may be heterogeneously distributed on the tubular body portion of an intraluminal prosthesis.

According to embodiments of the present invention, virtually any type of medical device may have a portion that is formed from PFPE material, or is coated with PFPE material. Exemplary medical devices include, but are not limited to, adaptors, applicators, aspirators, bandages, bands, blades, brushes, burrs, cables and cords, calipers, carvers, cases and containers, catheters, chisels, clamps, clips, condoms, connectors, cups, curettes, cutters, defibrillators, depressors, dilators, dissectors, dividers, drills, elevators, excavators, explorers, fasteners, files, fillers, forceps, gauges, gloves, gouges, handles, holders, knives, loops, mallets, markers, mirrors, needles, nippers, pacemakers, patches, picks, pins, plates, pliers, pluggers, probes, punches, pushers, racks, reamers, retainers, retractors, rings, rods, saws, scalpels, scissors, scrapers, screws, separators, spatulas, spoons, spreaders, stents, syringes, tapes, trays, tubes and tubing, tweezers, and wires.

According to embodiments of the present invention, PFPE material may be used to hermetically seal implantable electronic devices. For example, a housing of an implantable electronic device that contains one or more electronic components therein can be sealed with PFPE material to deter the ingress of moisture and foreign material into the housing when the electronic device is implanted within the body of a subject.

According to embodiments of the present invention, percutaneously implanted devices, including, but not limited to, defibrillators, pacemakers, cardiac resynchronization therapy devices, etc., have surfaces formed from and/or coated with PFPE material. For example, an implantable intravascular electrophysiological device that may carry out cardioversion, pacing and/or defibrillation of a heart includes a pulse generator and one or more electrodes electrically coupled to the pulse generator. The pulse generator includes a housing having a surface formed from and/or coated with PFPE material. The one or more electrodes may also have a coating of PFPE material. During implantation, the pulse generator is introduced into the vasculature of a subject, advanced to a desired vessel and anchored in place within the vessel. The electrode(s) are positioned within the heart or surrounding vessels as needed to deliver electrical pulses to the appropriate location. An anchor for retaining the device in the subject's vasculature may be a separate component or may be integrally formed with the intravascular electrophysiological device. The anchor has a surface formed from and/or coated with PFPE material.

The PFPE material of the pulse generator housing and anchor forms a smooth surface that prevents endothelial growth and thrombus formation thereon. The PFPE material may include one or more pharmacological agents configured to elute therefrom when the anchor is implanted within the vasculature of a subject. PFPE material is useful for any type of percutaneously implanted devices because it has low thrombogenic potential, low protein absorption, low surface energy, and high lubricity and flexibility.

An intravascular electrophysiological device, according to embodiments of the present invention, may also include a liner that is configured to be deployed within the vessel of a subject prior to implantation of the pulse generator housing. The pulse generator housing is configured to be deployed within the liner after the liner has been deployed within the vessel. The liner is formed from and/or coated with PFPE material.

According to embodiments of the present invention, a method of forming a polymeric coating on an interior surface of a hollow organ or tissue lumen includes applying liquid PFPE material to an interior surface of a hollow organ or tissue lumen, and then curing the PFPE material to form an oxygen permeable, bacterial impermeable polymer coating on the surface.

According to embodiments of the present invention, a method of repairing in situ a defect (e.g., a defect caused by a surgical procedure, by trauma, etc.) in a lung within the body of a subject includes applying a patch comprising oxygen permeable, bacterial impermeable liquid PFPE material over the lung defect, and then curing the liquid PFPE material to seal the patch to adjacent lung tissue so as to prevent air leakage therethrough. The patch may be applied in various ways including spraying liquid PFPE material onto lung tissue. Alternatively, the patch may be a preformed patch. According to embodiments of the present invention, the patch may include various materials including, but not limited to, collagen, gelatin, albumin, fibrin and elastin.

According to embodiments of the present invention, a method of implanting an arterio-venous shunt within the body of a subject includes implanting a mold within the body of a subject, wherein the mold is configured to form a tubular body, injecting an oxygen permeable, bacterial impermeable liquid PFPE material into the mold, curing the liquid PFPE material to form a tubular body, and connecting the tubular body to blood vessels in the body to form a shunt therebetween. The PFPE material may include one or more pharmacological agents, and may be configured to allow the one or more pharmacological agents to elute therefrom when the shunt is deployed within a body of a subject.

According to embodiments of the present invention, a method of implanting an arterio-venous shunt within the body of a subject includes implanting a tubular body comprising oxygen permeable, bacterial impermeable PFPE material within the body of a subject, and then connecting the tubular body to blood vessels in the body to form a shunt therebetween. The tubular body may include one or more pharmacological agents and the PFPE material of the tubular body is configured to allow the one or more pharmacological agents to elute therefrom when the shunt is deployed within a body of a subject.

According to embodiments of the present invention, a method of forming an arterio-venous shunt within the body of a subject includes applying an oxygen permeable, bacterial impermeable liquid PFPE material onto a surface of an existing vessel within the body of a subject, wherein the vessel serves as a mold, and curing the liquid PFPE material to form an arterio-venous shunt.

According to embodiments of the present invention, a method of repairing an arterio-venous shunt within the body of a subject includes applying an oxygen permeable, bacterial impermeable liquid PFPE material onto a surface of a shunt within the body of a subject, and curing the liquid PFPE material.

According to embodiments of the present invention, a method of repairing in situ a defect in a passageway (e.g., trachea, esophagus, etc.) within the body of a subject includes applying a patch comprising oxygen permeable, bacterial impermeable liquid PFPE material over the defect, and curing the liquid PFPE material to seal the patch to adjacent tissue so as to prevent leakage therethrough. According to embodiments of the present invention, applying a patch may include spraying liquid PFPE material onto tissue of the passageway. According to embodiments of the present invention, a patch may be a preformed patch. According to embodiments of the present invention, the PFPE material may include one or more pharmacological agents for treating the passageway.

According to embodiments of the present invention, an artificial tissue material for use within the lungs of a patient comprises a membrane of PFPE material that simulates alveolar action.

According to embodiments of the present invention, a material for use within a heart-lung machine, comprises a membrane of PFPE material that enhances gas exchange during artificial respiration.

According to embodiments of the present invention, an intraocular implant comprises oxygen permeable, bacterial impermeable liquid PFPE material.

According to embodiments of the present invention, a contact lens comprises oxygen permeable, bacterial impermeable liquid PFPE material.

According to embodiments of the present invention, a cochlear implant comprises oxygen permeable, bacterial impermeable liquid PFPE material.

According to embodiments of the present invention, a method of treating tissue within a body of a subject includes encapsulating tissue with liquid PFPE material, and curing the PFPE material to form an oxygen permeable, bacterial impermeable polymer coating on the tissue.

According to embodiments of the present invention, a method of treating tissue within the body of a subject includes forming a passageway in tissue within the body of a subject, inserting liquid PFPE material in the passageway, and curing the PFPE material to form an oxygen permeable, bacterial impermeable polymer material that facilitates growth of the tissue and enhances viability of surrounding tissues during healing and angiogenic phase. For example, the tissue may be heart muscle tissue and the PFPE material may facilitate revascularization of the heart muscle tissue. According to embodiments of the present invention, the steps of inserting and curing PFPE material may be performed as part of a transmyocardial revascularization procedure. The PFPE material may include one or more pharmacological agents for treating the tissue.

According to embodiments of the present invention, a method of promoting tissue growth within the body of a subject includes applying liquid PFPE material to tissue, and curing the PFPE material to form an oxygen permeable, bacterial impermeable polymer material that facilitates growth of the tissue. The PFPE material may include one or more pharmacological agents for treating the tissue.

According to embodiments of the present invention, a method of producing fabric includes coating a fabric with liquid PFPE material, and curing the liquid PFPE material to form a fabric having low surface energy. Exemplary fabrics include, but are not limited to, polytetrafluoroethylene, polyamides, polyesters, polyolefins, and Lycra. According to embodiments of the present invention, the fabric may comprise non-woven material.

According to embodiments of the present invention, parylene may be utilized as an underlying layer of material over which PFPE material is coated. According to embodiments of the present invention, any of the above-described items including, but not limited to implantable intravascular electrophysiological devices, orthopedic apparatus configured to be implanted within the body of a subject, bandages configured to be applied to the body of a subject, surgical sutures, artificial blood vessels, intraluminal prostheses, medical apparatus, implantable electronic devices, artificial tissue material for use within lungs, material for use within a heart-lung machine, intraocular implants, contact lenses, cochlear implants, etc., may include a parylene underlayer and a coating of PFPE material.

PFPE materials may also be utilized in repairing blood vessel aneurysms. According to embodiments of the present invention, a liquid PFPE material is injected into an aneurysm within a blood vessel and then cured as described above. The cured PFPE material serves as a filler that prevents the aneurysm from bursting.

According to other embodiments of the present invention, PFPE materials may be utilized to treat colonic diverticulosis. For example, a herniation in the colon wall of a subject is filled with a liquid PFPE material and then cured as described above. The cured PFPE material seals the herniation and prevents a rupture thereof.

In each of the embodiments described herein, curing of liquid PFPE may be performed by exposing the liquid PFPE material to heat, light, or other radiation (e.g., microwave radiation, infrared radiation, etc.). In addition, curing initiators that facilitate curing may be added to liquid PFPE material. Also, in embodiments where liquid PFPE material is applied within the body of a subject, the curing of the liquid PFPE material may be monitored via any of various known techniques including, but not limited to, magnetic resonance imaging (MRI), X-ray fluoroscopy, and ultrasound imaging.

### DETAILED DESCRIPTION OF THE INVENTION

The present invention now is described more fully hereinafter with reference to the accompanying drawings, in which embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

The term “biocompatible” as used herein, is intended to denote a material that, upon contact with a living element such as a cell or tissue, does not cause toxicity.

The term “eluting” is used herein to mean the release of a pharmacological agent from a polymeric material. Eluting may also refer to the release of a material from a substrate via diffusional mechanisms or by release from a polymeric material/substrate as a result of the breakdown or erosion of the material/substrate.

The term “erodible” as used herein refers to the ability of a material to maintain its structural integrity for a desired period of time, and thereafter gradually undergo any of numerous processes whereby the material substantially loses tensile strength and mass. Examples of such processes comprise enzymatic and non-enzymatic hydrolysis, oxidation, enzymatically-assisted oxidation, and others, thus including bioresorption, dissolution, and mechanical degradation upon interaction with a physiological environment into components that the patient's tissue can absorb, metabolize, respire, and/or excrete. The terms “erodible” and “degradable” are intended to be used herein interchangeably.

The term “fluoropolymer,” as used herein, has its conventional meaning in the art. See generally Fluoropolymers (L. Wall, Ed. 1972) (Wiley-Interscience Division of John Wiley & Sons); see also Fluorine-Containing Polymers, 7 Encyclopedia of Polymer Science and Engineering 256 (H. Mark et al. Eds., 2d Ed. 1985). The formation of fluoropolymers are described in U.S. Pat. Nos. 5,922,833; 5,863,612; 5,739,223; 5,688,879; and 5,496,901 to DeSimone, each of which is incorporated herein by reference in its entirety.

The term “hydrophobic” is used herein to mean not soluble in water.

The term “hydrophilic” is used herein to mean soluble in water.

The term “lumen” is used herein to mean any inner open space or cavity of a body passageway.

The terms “polymer” and “polymeric material” are synonymous and are to be broadly construed to include, but not be limited to, homopolymers, copolymers, terpolymers, and the like.

The term “prosthesis” is used herein in a broad sense to denote any artificial device used to replace a body part. An intraluminal prosthesis is a device which is implanted in the body of a subject for some therapeutic reason or purpose including, but not limited to, stents, drug delivery devices, etc.

The term “subject” is used herein to describe both human beings and animals (e.g., mammalian subjects) for medical, veterinary, testing and/or screening purposes.

The term “toxic materials” is intended to include all types of foreign materials, contaminants, chemicals, physical impurities, and the like, without limitation, that may be harmful to a subject.

As used herein, phrases such as “between X and Y” and “between about X and Y” should be interpreted to include X and Y.

As used herein, phrases such as “between about X and Y” mean “between about X and about Y.”

As used herein, phrases such as “from about X to Y” mean “from about X to about Y.”

As used herein, the term “and/or” includes any and all combinations of one or more of the associated listed items.

According to embodiments of the present invention, liquid curable perfluoropolyether (PFPE) materials, and derivatives therefrom, are provided for use as coatings, sealants, flexible fillers, structural parts, etc., and in a wide variety of medical applications, particularly where silicone has been utilized conventionally. Hereinafter, the term “PFPE material” shall include all perfluoropolyethers and all derivatives therefrom.

PFPE materials are a unique class of fluoropolymers that are liquids at room temperature, exhibit low surface energy, low modulus, high gas permeability, high lubricity, and low toxicity with the added feature of being extremely chemically resistant. PFPE materials are particularly advantageous for use in medical applications because PFPE materials are oxygen permeable, but impermeable to many pathogens. The synthesis of PFPE materials is described generally in W. C. Bunyard et al., Macromolecules 32, 8224 (1999), which is incorporated by reference in its entirety. In general, fluoropolyethers are polymeric compounds composed of multiple, sequentially linked, fluorinated aliphatic ether units (e.g., polymers of the formula (RO)ₙ R wherein the R groups are the same or different and are linear or branched, saturated or unsaturated C₁-C₄ alkyl; typically linear or branched saturated C₁-C₄ alkyl, with the number of repeats “n” giving the desired molecular weight); perfluoropolyether are such polymers in which essentially all of the hydrogens have been substituted with fluorine. Examples of perfluoropolyethers are illustrated below in Table 1 and include perfluoropolymethyl-isopropyl-ethers such as: (i) polymers marketed under the tradename FOMBLIN®; (ii)polymers marketed under the tradename AFLUNOX®, and (iii) polymers marketed under the tradename FOMBLIN Z_DOL™. See, e.g., U.S. Pat. No. 6,582,823, which is incorporated herein by reference in its entirety.

Table 1

TABLE 1

| | | |
|---|---|---|
| Krytox ® | DuPont | 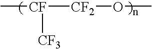 |
| Fomblin ® Y | Ausimont | 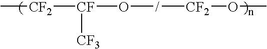 |
| Fomblin ® Z | Ausimont | 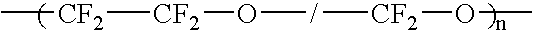 |
| Demnum ® | Daikin | 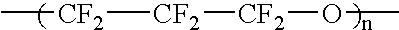 |

The synthesis and photocuring of these materials can be done in a manner similar to that based on earlier work done by Bongiovanni et al., which is described in Macromol. Chem. Phys. 198, 1893 (1997) and which is incorporated by reference in its entirety. The reaction involves the methacrylate-functionalization of a commercially available PFPE diol (Mₙ=3,800 g/mol) with isocyanato-ethyl methacrylate. Subsequent photocuring of the material is accomplished by blending it with 1 wt % of 2,2-dimethoxy-2-phenylacetophenone (DMPA) and exposing it to UV radiation (A=365 nm) as illustrated below in Table 2.

TABLE 2
Crosslinked PFPE Network

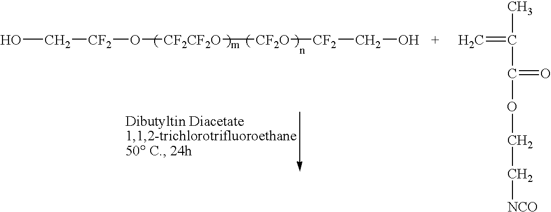

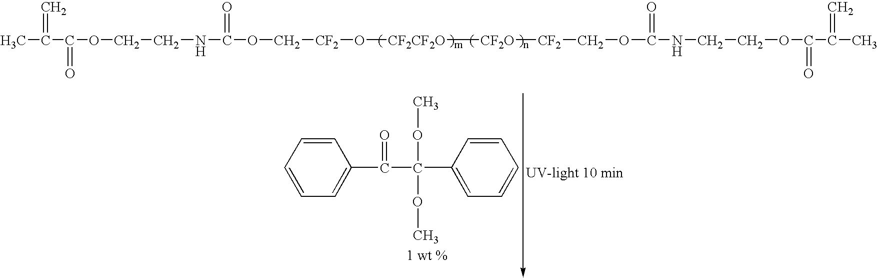

PFPE materials may also be functionalized with various groups, such as with epoxy groups, vinyl groups, hydroxyl groups, isocyanate groups, and amino groups and subsequently cured via various curing mechanisms well known to those skilled in the art including, but not limited to, radical, urethane, epoxy, and cationic curing mechanisms. Examples of radical curing include thermal curing with added free radical initiators, such as azo initiators, peroxides, acyl peroxides, and peroxy dicarbonates. Examples of radical curing also include photochemical curing with added photo-generated free radical initiators such as 2,2-dimethoxy-2-phenylacetophenone. Epoxy containing PFPE materials may be cured via the addition of amines or by cationic ring-opening methods. Examples of amines useful for curing epoxy containing PFPE materials include 4,4′-diaminodiphenylsulfone. Examples of cationic ring-opening methods for curing epoxy containing PFPE materials include the use of non-ionic or ionic photoacid generators. Useful nonionic photoacid generators include 2,5-dinitrobenzyl tosylate or 2-perfluorohexyl-6-nitrobenzyl tosylate. Useful ionic photoacid generators include diphenyliodium tetraphenyl borate or diphenyliodonium tetra-[3,5-bis(trifluoromethyl) phenyl] borate. Urethane curing mechanisms may include isocyanate reactions with hydroxyl or amine compounds.

PFPE materials according to embodiments of the present invention can be modified and “tuned” to achieve various characteristics and functionalities. For example, reactive monomers can be added to PFPE materials to adjust physical properties including, but not limited to, modulus, wetting, various surface characteristics, etc. Reactive monomers that can be added to modify the properties can include styrenics such as styrene, and para-chloromethylstyrene, t-butylstyrene and divinylbenzene; alkyl (meth)acrylates such as butyl acrylate and methyl methacrylate; functional (meth)acrylates such as hydroxyethylmethacrylate, acryloxyethyltrimethylammonium chloride (AETMAC), hydroxyethylacrylate (HEA), cyanoacrylates, fluoroalkyl (meth)acrylates, 2-isocyanatoethyl methacrylate, glycidyl methacrylate, allyl methacrylate and poly(ethylene glycol)diacrylate (PEGdiA); olefins such as norbornene, vinylacetate, 1-vinyl-2-pyrrolidone, and alkylacrylamides.

In addition, various additives can be added to PFPE materials according to embodiments of the present invention including, but not limited to, pharmacological agents, fillers, bioerodible materials, porogens, deoxyribonucleic acid (DNA), oligonucleotides, peptides, growth hormones, etc. Mechanical fillers that may be added to PFPE materials according to embodiments of the present invention may include, but are not limited to, silica, clay, and other materials of various sizes (e.g., nanoparticles). Additives can be included with PFPE material in various ways including, but not limited to, being chemically attached to PFPE material, being embedded within PFPE material, being dispersed in PFPE material, etc. The term “attached”, as used herein, encompasses all methods of adding additives to PFPE materials.

In addition, PFPE materials can be tuned to cure as a rigid structure, as a flexible structure, and/or as a partially rigid and partially flexible structure. Moreover, the degree of rigidity and flexibility can also be designed into the PFPE material via additives.

In addition, embodiments of the present invention may utilize composite materials having variable layers of rigid and less rigid PFPE materials. For example, layers of uniaxially and biaxially oriented materials may be utilized such that anisotropic properties can be obtained (e.g., flexibility in one direction and strength or rigidity in another direction, etc.).

In general, pharmacological agents suitable for use with PFPE materials (and according to embodiments of the present invention) include, but are not limited to, drugs and other biologically active materials, and may be intended to perform a variety of functions, including, but not limited to: anti-cancer treatment (e.g., Resan), anti-clotting or anti-platelet formation, the prevention of smooth muscle cell growth, migration, and proliferation within a vessel wall. According to embodiments of the present invention, pharmacological agents suitable for use with PFPE materials include, but are not limited to, antineoplastics, antimitotics, antiinflammatories, antiplatelets, anticoagulants, antifibrins, antithrombins, antiproliferatives, antibiotics, antioxidants, and antiallergic substances as well as combinations thereof. Examples of antineoplastics and/or antimitotics include paclitaxel (cytostatic and ant-inflammatory) and it's analogs and all compounds in the TAXOL® (Bristol-Myers Squibb Co., Stamford, Conn.) family of pharmaceuticals, docetaxel (e.g., TAXOTERE® from Aventis S. A., Frankfurt, Germany) methotrexate, azathioprine, vincristine, vinblastine, fluorouracil, doxorubicin hydrochloride (e.g., ADRIAMYCIN® from Pharmacia & Upjohn, Peapack, NJ), and mitomycin (e.g., MUTAMYCIN® from Bristol-Myers Squibb Co., Stamford, Conn.). Examples of antiinflammatories include Sirolimus and analogs thereof (including but not limited to Everolimus and all compounds in the Limus family of pharmaceuticals), glucocorticoids such as dexamethasone, methylprednisolone, hydrocortisone and betamethasone and non-steroidal antiinflammatories such as aspirin, indomethacin and ibuprofen. Examples of antiplatelets, anticoagulants, antifibrin, and antithrombins include sodium heparin, low molecular weight heparins, heparinoids, hirudin, argatroban, forskolin, vapiprost, prostacyclin and prostacyclin analogues, dextran, D-phe-pro-arg-chloromethylketone (synthetic antithrombin), dipyridamole, glycoprotein IIb/IIIa platelet membrane receptor antagonist antibody, recombinant hirudin, and thrombin inhibitors such as Angiomax™ (Biogen, Inc., Cambridge, Mass.) Examples of cytostatic or antiproliferative agents or proliferation inhibitors include everolimus, actinomycin D, as well as derivatives and analogs thereof (manufactured by Sigma-Aldrich, Milwaukee, Wis.; or COSMEGEN® available from Merck & Co., Inc., Whitehouse Station, NJ), angiopeptin, angiotensin converting enzyme inhibitors such as captopril (e.g., CAPOTEN® and CAPOZIDE® from Bristol-Myers Squibb Co., Stamford, Conn.), cilazapril or lisinopril (e.g., Prinivilo and PRINZIDE® from Merck & Co., Inc., Whitehouse Station, NJ); calcium channel blockers (such as nifedipine), colchicine, fibroblast growth factor (FGF) antagonists, fish oil (omega 3-fatty acid), histamine antagonists, lovastatin (an inhibitor of HMG-CoA reductase, a cholesterol lowering drug, brand name MEVACOR® from Merck & Co., Inc., Whitehouse Station, NJ), monoclonal antibodies (such as those specific for Platelet-Derived Growth Factor (PDGF) receptors), nitroprusside, phosphodiesterase inhibitors, prostaglandin inhibitors, suramin, serotonin blockers, steroids, thioprotease inhibitors, triazolopyrimidine (a PDGF antagonist), and nitric oxide. An example of an antiallergic agent is permirolast potassium. Other therapeutic substances or agents that may be used include alphainterferon, genetically engineered epithelial cells, and dexamethasone.

Pain relief agents may also be added to PFPE materials according to embodiments of the present invention.

According to embodiments of the present invention, PFPE materials may be tuned such that, when cured, the PFPE material is contiguous, porous, and/or biphasic. Porous or biphasic materials can be achieved by adding other components that will phase separate such as salts (e.g., sodium chloride); sugars such as sucrose; water or saline solutions; other polymers such as polyethylene glycols, poly(vinyl alcohol, or biodegradable polymers such as polylactides, polyglycolides, polycaprolactone; or added gases or gases that are generated in situ such as through the addition of water to isocynate compounds which releases CO₂.

According to embodiments of the present invention, PFPE materials may be applied neat or by using a solvent to facilitate the coating process prior to curing. Any solvent which can dissolve the PFPE materials is useful. The solvent can reduce the viscosity of the PFPE materials to facilitate the coating process. A lower viscosity can enable the formation of contiguous films or facilitate the formation of thinner films. Exemplary solvents include fluorinated solvents such as FLUORINERT® manufactured by 3M Company (St. Paul, Minn.).

According to embodiments of the present invention, PFPE materials may be used in any application where silicone materials have conventionally been used. For example, PFPE materials may be utilized in coatings, sealants, adhesives, structural parts, fillers, implants, etc.

PFPE materials, according to embodiments of the present invention, may be utilized in virtually any medical application, product and method. According to embodiments of the present invention, curing of PFPE material(s) in the various applications described herein may be accomplished in various ways including, but not limited to, the use of heat, light and/or other electromagnetic radiation (e.g., microwave, infrared, etc.).

The following sections describe a few exemplary embodiments of the present invention. These examples are not intended to encompass the entire scope of embodiments of the present invention.

Orthopedic Applications

PFPE materials may be used in various orthopedic applications, including orthopedic devices and implants, as well as orthopedic surgical procedures. Embodiments of the present invention facilitate building and providing new devices and structures for placement within the body of a subject, in addition to rebuilding and repairing existing devices and structures in situ. For example, PFPE materials may be utilized in building new hip joints and in repairing existing hip joints (e.g., an original hip joint or a replacement hip joint) in situ. The high wear, high lubricity properties of PFPE are particularly beneficial for hip joints. The hip joint ball and socket can be made out of PFPE material or the ball and socket surfaces of a metallic implant can be coated with PFPE material.

According to embodiments of the present invention, a method of repairing skeletal or skeletal-related (e.g., ligaments, tendons, cartilage, muscles, etc.) damage within the body of a subject includes inserting and positioning an enclosure is adjacent (e.g., within, next to, on top of, etc.) the damaged skeletal portion (or skeletal-related portion) of a subject, injecting a liquid PFPE material into the enclosure, and curing the liquid PFPE material. Such an enclosure may be made of durable polymers (which would be removed post cure) such as PE, PET, polycarbonate etc or erodible materials (which would not require removal) such as poly(L-lactide) or its radiosiomers, poly glycolic acid, polyanhydrides etc. Enclosures or molds are inserted minimally invasively or surgically.

Curing the liquid PFPE material may be performed in various ways. For example, the liquid PFPE material may be exposed to heat, light or other radiation. For example, localized exposure to light may be provided by fiber optics, “light pipes”, etc. Localized exposure to radiation may be provided by devices capable of delivering a directed beam of radiation. In addition, curing initiators may be added to the liquid PFPE material.

The cured PFPE material forms a rigid structure that provides structural support to the skeletal portion of the subject. For example, the damage may be a crack or other defect in a bone and the enclosure is positioned within the crack. The liquid PFPE material, upon curing, seals the crack and provides structural support to the bone. Alternatively, depending on the functionality of the PFPE material, the PFPE material (or one or more portions of the PFPE material) upon curing may remain flexible. Accordingly, the cured, flexible PFPE material may replace portions of ligaments, tendons, cartilage, muscles, etc. and other flexible tissues within the body of a subject.

According to other embodiments of the present invention, a damaged skeletal portion may be a damaged spinal component, such as discs and vertebral bodies. In an application of the present invention, an enclosure as described above may be inserted within the nuclear space of a vertebral body. The liquid PFPE material injected therein, upon curing, mimics a native, healthy nucleus and restores normal vertebral function by preventing denaturization of cells and failure of the annular portion of the disc.

According to other embodiments of the present invention, the skeletal portion may be a joint having a damaged portion. Any joint in the body of a subject may be repaired in accordance with embodiments of the present invention including, but not limited to, hips, knees, ankles, phalange joints, elbows, and wrists.

According to an embodiment of the present invention, a joint may have a damaged wear surface. Liquid PFPE material is applied to the damaged wear surface and, upon curing, provides a repaired wear surface.

According to other embodiments of the present invention, PFPE materials may be utilized in conjunction with, or in place of, arthroscopic surgery to repair a damaged joint. Unwanted material (e.g., damaged cartilage, etc.) is removed from a joint and an enclosure as described above is positioned at the location of the unwanted material. Liquid PFPE material is injected into the enclosure and cured. The cured PFPE material serves as a replacement for the original structure or surface.

According to other embodiments of the present invention, an implantable orthopedic apparatus has an outer surface of oxygen permeable, bacterial impermeable PFPE material. The implantable apparatus may be formed from the PFPE material and/or the PFPE material may be a coating on the apparatus.

Implantable orthopedic apparatus according to embodiments of the present invention may be artificial or may be cadaver parts refurbished using PFPE materials. For example, a knee from a cadaver can be refurbished as described above to improve wear surfaces and to repair damaged areas, etc. Elastic moduli that can be achieved for cured and modified PFPE based materials can range from 1 MPa to 2 GPa.

Dermatological Applications

PFPE materials are particularly advantageous for use in various dermatological applications including, but not limited to, bandages, dressings and wound healing applications, burn care, reconstructive surgery, surgical glue, sutures, etc. Because PFPE materials are oxygen permeable and bacterial impermeable, tissue underlying a PFPE bandage can receive oxygen while being protected against the ingress of dirt, bacteria, microbial organisms, pathogens and other forms of contamination and toxicity. Moreover, PFPE materials are non-toxic. In addition, the oxygen permeability and carrying capacity of PFPE materials can also help with preventing necrosis of healthy tissue under bandages and dressings, or under an area being treated.

According to an embodiment of the present invention, a method of applying “instant skin” to the body of a subject includes applying an oxygen permeable, bacterial impermeable liquid PFPE material onto a portion of the body of a subject, and curing the PFPE material to form a protective bandage that facilitates healing of the underlying tissue. The protective bandage is antiseptic, flexible, waterproof and lets the underlying skin breathe (i.e., it forms a film that is oxygen permeable, but bacteria impermeable).

The liquid PFPE material can be applied in various ways including, but not limited to, spraying, swabbing, etc. As described above, curing can be performed in various ways including, but not limited to, exposing the liquid PFPE material to light, heat and/or other radiation. Curing may be facilitated by adding curing initiators to the liquid PFPE material.

According to other embodiments of the present invention, PFPE materials can be modified to include adhesive properties so that the PFPE material can serve the function of a non-toxic, curable liquid bandage for sealing wounds. Exemplary material that can be added to PFPE materials to achieve adhesiveness includes cyanoacrylate. When cured, the PFPE material is flexible, yet remains adhered to moving parts such as knees and elbows. In addition, bandages formed from this material provide barriers to infection, can reduce pain to the wearer because of lower surface energy, and can control bleeding better than traditional bandages.

According to embodiments of the present invention, PFPE materials can be utilized in adhesion prevention products for various post-surgical tissue applications. For example, PFPE material can be applied to post-surgical tissue to prevent other materials and tissue from adhering to the post-surgical tissue. PFPE material may be applied in post-lung lobectomy, hysterectomy, appendectomy, hernia repair or any application where tissue has been injured and connective growth to surrounding tissues or organs is not desired.

Cardiovascular and Intraluminal Applications

PFPE materials according to embodiments of the present invention may be used in various cardiovascular applications and in various other intraluminal applications, including devices and methods. According to embodiments of the present invention, PFPE oils may be used as synthetic blood and/or blood substitutes. Moreover, PFPE materials according to embodiments of the present invention may be utilized in blood analysis and treatment devices.

According to other embodiments of the present invention, artificial blood vessels having oxygen permeable, bacterial impermeable PFPE materials can be produced for replacing damaged and/or occluded vessels within the body of a subject. Not only can PFPE materials serve as conduits for blood flow, but they also can allow for diffusion of oxygen and nutrients through the vessel wall into surrounding tissues thus functioning much like a normal healthy blood vessel to various areas of the body of a subject.

According to embodiments of the present invention, a method of replacing in situ a portion of a blood vessel within the body of a subject includes injecting an oxygen permeable, bacterial impermeable liquid PFPE material into a lumen of a portion of a blood vessel to form an artificial blood vessel. The blood vessel portion serves as a mold for forming the artificial vessel. The PFPE material is then subjected to conditions sufficient to cure the PFPE material such that a working replacement for the blood vessel portion is produced. Curing may be performed in various ways as described above. The original blood vessel portion may be removed from the body of the subject. If the blood vessel portion being replaced is occluded or partially occluded, the occluding material is removed prior to injecting the liquid PFPE material into the lumen.

According to embodiments of the present invention, replacement blood vessels (as well as other cardiovascular vessels) incorporating PFPE materials can be produced ex vivo for subsequent surgical implantation within the body of a subject.

Embodiments of the present invention are particularly advantageous regarding repair and/or replacement of blood vessels. Given their high oxygen carrying ability and permeability, artificial vessels formed from PFPE materials according to embodiments of the present invention have highly functional properties with synthetic vasavasorum characteristics. PFPE materials allow diffusion of oxygen through the walls and into surrounding dependent tissues, allow diffusion of sustaining nutrients, diffusion of metabolites. PFPE materials mimic vessels mechanically as they are flexible and compliant. Moreover, embodiments of the present invention are particularly suitable for use in heart by-pass surgery and as artificial arterio-venous shunts. PFPE materials can also be used to repair natural or synthetic a-v shunts by coating the inside surface of the damaged or worn vessel and curing as previously described.

PFPE materials according to embodiments of the present invention may be utilized in various intraluminal applications including, but not limited to, stents (and other tissue scaffolding devices), catheters, heart valves, electrical leads associated with rhythm management, balloons and other angioplasty devices, drug delivery devices, etc. Moreover, PFPE materials according to embodiments of the present invention may be embodied in the material(s) of these devices or in coatings on these devices. Intraluminal prostheses provided in accordance with embodiments of the present invention may be employed in sites of the body other than the vasculature including, but not limited to, biliary tree, esophagus, bowels, tracheo-bronchial tree, urinary tract, etc.

According to embodiments of the present invention, percutaneously implanted devices including, but not limited to, pacemakers, defibrillatory and implanted cardioverter defibrillatory (“ICD”) devices, utilized for treatment of heart rhythm conditions, may be formed from and/or coated with PFPE material(s). As known to those skilled in the art, a pacemaker is implanted in patients who have bradycardia (slow heart rate). The pacemaker detects periods of bradycardia and delivers electrical stimuli to increase the heartbeat to an appropriate rate. ICDs are implanted in patients who may suffer from episodes of fast and irregular heart rhythms called tachyarrhythmias. An ICD can cardiovert the heart by delivering electrical current directly to the heart to terminate an atrial or ventricular tachyarrhythmia, other than ventricular fibrillation. An ICD may alternatively defibrillate the heart in a patient who may suffer ventricular fibrillation (VF), a fast and irregular heart rhythm in the ventricles. During a VF episode, the heart quivers and can pump little or no blood to the body, potentially causing sudden death. An ICD implanted for correction of ventricular fibrillation can detect a VF episode and deliver an electrical shock to the heart to restore the heart's electrical coordination.

Another type of implantable defibrillation device that may be formed from and/or coated with PFPE material, according to embodiments of the present invention, treats patients who may suffer from atrial fibrillation (AF), which is a loss of electrical coordination in the heart's upper chambers (atria). During AF, blood in the atria may pool and clot, placing the patient at risk for stroke. An electrophysiological device implanted for correction of atrial fibrillation can detect an AF episode and deliver an electrical shock to the atria to restore electrical coordination. Exemplary implantable electrophysiological devices and systems that may be formed from and/or coated with PFPE material(s) according to embodiments of the present invention are described in PCT Publication No. WO 2005/000398, entitled Intravascular Electrophysiological System and Methods, and is incorporated herein by reference in its entirety.

According to embodiments of the present invention, an implantable intravascular electrophysiological device that may carry out cardioversion, pacing and/or defibrillation of a human heart includes a pulse generator and one or more electrodes electrically coupled to the pulse generator. The pulse generator produces electrical pulses which are delivered via the one or more electrodes to various portions of a blood vessel of a subject. The pulse generator includes a housing having a contour that allows blood flow through the vessel when implanted within the blood vessel with as little obstruction as possible. The pulse generator housing is formed from or has a coating of PFPE material. The one or more electrodes may also be formed from or have a coating of PFPE material. During implantation, the pulse generator is introduced into the vasculature of a subject, advanced to a desired vessel and anchored in place within the vessel. The electrode(s) are positioned within the heart or surrounding vessels as needed to deliver electrical pulses to the appropriate location.

According to embodiments of the present invention the pulse generator housing PFPE material may include one or more pharmacological agents configured to elute therefrom when the intravascular electrophysiological device is implanted within a blood vessel of a subject.

PFPE materials utilized in various devices of the present invention are advantageous over conventional materials because PFPE material has low thrombogenic potential, low protein absorption, low surface energy, and high lubricity and flexibility.

An intravascular electrophysiological device, according to embodiments of the present invention, may also include an anchor for retaining the device in a subject's vasculature (e.g., in the superior vena cava, inferior vena cava, left or right subclavian, etc.). Various types of anchors may be utilized, without limitation. Moreover, an anchor may be a separate component or may be integrally formed with the intravascular electrophysiological device. The anchor is coated with or formed from PFPE material. The PFPE material forms a smooth surface that prevents endothelial growth and thrombus formation thereon. The PFPE material may include one or more pharmacological agents configured to elute therefrom when the anchor is implanted within the vasculature of a subject.

An intravascular electrophysiological device, according to embodiments of the present invention, may also include a liner that is configured to be deployed within the vessel of a subject prior to implantation of the pulse generator housing. The pulse generator housing is configured to be deployed within the liner after the liner has been deployed within the vessel. The liner is formed from and/or coated with PFPE material. The liner typically has a length that is greater than a length of the pulse generator housing.

An intravascular electrophysiological device, according to embodiments of the present invention, may also include a removable implantation tool (e.g., mandrels, stylets, guidewires, etc.) that is configured to facilitate implantation of the pulse generator housing within the vasculature of a subject, as would be understood by those skilled in the art. The removable implantation tool may be formed from and/or coated with a PFPE material.

Stents are typically used as adjuncts to percutaneous transluminal balloon angioplasty procedures, in the treatment of occluded or partially occluded arteries and other blood vessels. As an example of a balloon angioplasty procedure, a guiding catheter or sheath is percutaneously introduced into the cardiovascular system of a patient through, for example, the femoral arteries and advanced through the vasculature until the distal end of the guiding catheter is positioned at a point proximal to the lesion site. A guidewire and a dilatation catheter having a balloon on the distal end are introduced through the guiding catheter with the guidewire sliding within the dilatation catheter. The guidewire is first advanced out of the guiding catheter into the patient's vasculature and is directed across the arterial lesion. The dilatation catheter is subsequently advanced over the previously advanced guidewire until the dilatation balloon is properly positioned across the arterial lesion. Once in position across the lesion, the expandable balloon is inflated to a predetermined size with a radiopaque liquid at relatively high pressure to radially compress the atherosclerotic plaque of the lesion against the inside of the artery wall and thereby dilate the lumen of the artery. The balloon is then deflated to a small profile so that the dilatation catheter can be withdrawn from the patient's vasculature and blood flow resumed through the dilated artery.

Balloon angioplasty sometimes results in short or long term failure (restenosis). That is, vessels may abruptly close shortly after the procedure or restenosis may occur gradually over a period of months thereafter. To counter restenosis following angioplasty, implantable intraluminal prostheses, commonly referred to as stents, are used to achieve long term vessel patency. A stent functions as scaffolding to structurally support the vessel wall and thereby maintain luminal patency, and are transported to a lesion site by means of a delivery catheter.

Types of stents may include balloon expandable stents, spring-like, self-expandable stents, and thermally expandable stents. Balloon expandable stents are delivered by a dilitation catheter and are plastically deformed by an expandable member, such as an inflation balloon, from a small initial diameter to a larger expanded diameter. Self-expanding stents are formed as spring elements which are radially compressible about a delivery catheter. A compressed self-expanding stent is typically held in the compressed state by a delivery sheath. Upon delivery to a lesion site, the delivery sheath is retracted allowing the stent to expand. Thermally expandable stents are formed from shape memory alloys which have the ability to expand from a small initial diameter to a second larger diameter upon the application of heat to the alloy.

PFPE materials, according to embodiments of the present invention, may be used with all of the above-described cardiovascular and intraluminal devices. PFPE materials may be utilized in the material(s) of these devices and/or may be provided as a coating on these devices.

It may be desirable to provide localized pharmacological treatment of a vessel at the site being supported by a stent or other intraluminal device. Thus, sometimes it is desirable to utilize a stent both as a support for a lumen wall as a well as a delivery vehicle for one or more pharmacological agents. PFPE materials according to embodiments of the present invention may be configured to carry and release pharmacological agents. PFPE materials may be impregnated with pharmacological agents for delivery within a body of a subject. The impregnation of polymer materials is described in commonly assigned U.S. patent application Publication No.: 2004-0098106-A1, which is incorporated herein by reference in its entirety.

According to other embodiments of the present invention, liquid PFPE materials may be utilized in endoluminal sealing processes wherein the interior surfaces of tissue lumens are covered or sealed with polymeric material. Liquid PFPE materials are especially suitable for these procedures because of high lubricity and high permeability to oxygen. According to an embodiment of the present invention, a catheter or other instrument is configured to deliver liquid PFPE material to a tissue lumen and to cause the PFPE material to conform to the interior surface of the lumen. Upon curing, the PFPE material provides an improved interior surface. Lumen paving procedures and apparatus are described in U.S. Pat. Nos. 6,443,941; 5,800,538; 5,749,922; 5,674,287; and 5,213,580 to Slepian et al., each of which is incorporated herein by reference in its entirety.

According to embodiments of the present invention, PFPE materials may be incorporated into various types of patches utilized in lung surgical procedures. Patches according to embodiments of the present invention include spray-on patches wherein PFPE material is sprayed directly on lung tissue. Preformed patches configured to be attached and secured to lung tissue via conventional methods may also include PFPE material, according to embodiments of the present invention.

The use of a patch secured to lung tissue, such as over a wound from tumor removal or a rough surface of the lung, provides a seal to close the wound and prevent air leakage. Additionally, a patch incorporating PFPE materials may be used in conjunction with sutures and staples to provide additional sealing over the mechanical closures, for example, over the staple or suture line of a lobectomy. The oxygen carrying ability and permeability of PFPE materials makes them particularly suitable for use in lung repair. Moreover, because PFPE materials can be cured to a flexible state, they are particularly suitable for use as patches for lungs where expansion of a lung requires a flexible and strong bond with a gas-tight seal. According to embodiments of the present invention, PFPE materials may include one or more pharmacological agents that are configured to elute therefrom, as described above, when a patch is implanted within a subject's body.

According to embodiments of the present invention, PFPE materials can be utilized in arterio-venous (“AV”) shunts. As known to those skilled in the art, AV shunts are utilized to join an artery and vein, allowing arterial blood to flow directly into the vein. PFPE materials according to embodiments of the present invention can be utilized to repair AV shunts or create artificial ones, and this can be done both in vivo and ex vivo. According to embodiments of the present invention, PFPE materials may include one or more pharmacological agents that are configured to elute therefrom, as described above, when a shunt is implanted within a subject's body.

According to embodiments of the present invention, AV shunts utilized in dialysis treatment of patients may be replaced and/or repaired using PFPE materials. AV shunts implanted within dialysis patients periodically require replacement or repair. According to embodiments of the present invention, a damaged or worn AV shunt can be repaired in situ by coating the shunt with PFPE material and then curing the PFPE material as described above. According to other embodiments of the present invention, existing shunts can be removed and replaced with shunts containing PFPE materials.

According to embodiments of the present invention, PFPE material may be utilized in trachea and esophagus patches and repair procedures therefor. Patches according to embodiments of the present invention can be effective in preventing or reducing air leakage and/or food leakage from a damaged trachea and esophagus. Patches according to embodiments of the present invention may include spray-on patches wherein PFPE material is sprayed directly on trachea/esophagus tissue. Preformed patches configured to be attached and secured to trachea/esophagus tissue via conventional methods may also include PFPE material, according to embodiments of the present invention. According to embodiments of the present invention, PFPE materials may include one or more pharmacological agents that are configured to elute therefrom when a patch is implanted within a subject's body.

According to embodiments of the present invention, PFPE materials may be utilized as artificial lung material because they can enhance gas exchange during respiration. For example, PFPE materials may be utilized as substitute alveolar membrane material, both for an actual lung and for artificial lung machines and heart-lung machines. As known to those skilled in the art, the alveoli are components within the lung which facilitate oxygen/carbon dioxide exchange and the alveolus is a terminal sacule of an alveolar duct where gases are exchanged during respiration. The high oxygen exchange capacity of PFPE materials helps simulate the alveolar action of lung material, including alveoli and alveolus.

According to embodiments of the present invention, PFPE materials may be utilized in transmyocardial revascularization (TMR). As known to those skilled in the art, TMR is a procedure used to relieve severe angina or chest pain in very ill patients who are not candidates for bypass surgery or angioplasty. TMR involves drilling a series of holes from the outside or from the inside of the ventricles of the heart into the heart's pumping chamber, typically via a laser. These holes can stimulate the growth of new blood vessels (“revascularization”) and can destroy nerve fibers in the heart, thereby making a patient unable to feel chest pain.

According to embodiments of the present invention, PFPE materials can be injected into holes produced during a TMR procedure to facilitate revascularization of the heart tissue. Moreover, one or more pharmacological agents for facilitating revascularization, as well as for various other purposes, can be included with the PFPE material injected into the holes.

Vision and Hearing Applications

According to embodiments of the present invention, ocular implants and contact lenses are formed from PFPE material. These devices are advantageous over conventional ocular implants and contact lenses because the PFPE material is permeable to oxygen and resistant to bio-fouling. In addition, because of the lower surface energy, there is more comfort to the wearer because of lower friction. In addition, the refractive index of PFPE materials can be tuned (adjusted/precisely controlled) for optimum performance for ocular implants and contact lenses.

According to embodiments of the present invention, cochlear implants utilizing PFPE material are advantageous over implants formed from conventional materials. Utilizing PFPE material, tissue in-growth can be minimized, thus making removal of the device safer and less traumatic.

Tissue Treatment

According to embodiments of the present invention, liquid PFPE materials and blends thereof may be applied to various areas within the body of a subject. Upon curing, the PFPE material may serve as an oxygen permeable, bacterial impermeable protective coating. Moreover, oxygen-deprived tissue may be encapsulated with PFPE material. Tissue may also be replaced with PFPE material.

PFPE materials can be utilized for scaffolding for new tissue growth according to embodiments of the present invention. The high oxygen permeability of PFPE materials are particularly suitable for promoting tissue growth.

Other Devices, Systems and Tools

Various devices, including tools and implants, may incorporate PFPE material as described above. Exemplary devices include tubing, fabrics, filters, balloons, catheters, needles and other surgical tools, clamps and devices. These devices can be made from all types of materials including ceramics, glass, metals, polymers and composites thereof. The PFPE material may be used as coatings, adhesives, sealants or structural components or space-filling additives.

According to embodiments of the present invention, electronic devices configured to be implanted within the body of a subject are sealed with PFPE material. For example, a housing containing one or more electronic components therein may be hermetically sealed with PFPE material which prevents the ingress of moisture and bio-fouling into the housing when the electronics device is implanted within the body of a subject.

According to embodiments of the present invention, individual electronic components such as batteries, capacitors, etc. that are implanted within the body may be hermetically sealed via PFPE materials. PFPE materials can have high dielectric strength and thus can serve as very good electrical insulators.

According to embodiments of the present invention, medical tools and devices may be coated, sealed or comprised of PFPE material(s). Any type of medical instrument and device may be coated, sealed or comprised of PFPE material(s) including, but not limited to, instruments and devices utilized in cosmetic surgery, cardiology, dentistry and oral surgery, dermatology, ENT/otolaryngology, gynecology, laparoscopy, neurosurgery, orthopedics, ophthalmology, podiatry, urology, veterinary. The following is a non-exhaustive list of instruments and devices that may be coated, sealed or comprised of PFPE materials as described herein: adaptors, applicators, aspirators, bandages, bands, blades, brushes, burrs, cables and cords, calipers, carvers, cases and containers, catheters, chisels, clamps, clips, condoms, connectors, cups, curettes, cutters, defibrillators, depressors, dilators, dissectors, dividers, drills, elevators, excavators, explorers, fasteners, files, fillers, forceps, gauges, gloves, gouges, handles, holders, knives, loops, mallets, markers, mirrors, needles, nippers, pacemakers, patches, picks, pins, plates, pliers, pluggers, probes, punches, pushers, racks, reamers, retainers, retractors, rings, rods, saws, scalpels, scissors, scrapers, screws, separators, spatulas, spoons, spreaders, stents, syringes, tapes, trays, tubes and tubing, tweezers, and wires.

According to embodiments of the present invention, natural and synthetic fabrics and clothes may be coated, sealed and/or comprised of PFPE material(s). In particular, PFPE material(s) may be used to coat expanded polytetrafluoroethylene (also known as a GORETEX® membrane by W. L. Gore) materials and their derivatives and then cured. Other fabrics that can be coated include polyamides, polyesters, polyolefins, Lycra, etc. PFPE material(s) can make fabrics have a very low surface energy, and can change various fabrics performance properties. For example, a non-woven fabric of Nylon 6,6 can be coated with a PFPE material to produce a material having similar surface and barrier properties as a GORETEX® membrane, but at a reduced cost.

Tools and Systems for Applying, Curing, and Monitoring the Application and Curing of PFPE Materials

In addition to the materials and processes described above, embodiments of the current invention include the tools and systems required to deliver or use PFPE materials in medical devices and tools. This includes catheters; syringes; delivery cartridges for resins, curing agents; heat sources; light sources including directed light sources such as wands, light pipes and lasers and indirect light sources such as wide-area bulbs and arrays. These tools and systems can be used for the in situ delivery of PFPE materials or for the use or delivery of PFPE materials ex situ such as at a factory or custom manufacturing facility. Techniques can be used for monitoring or inspecting the delivery or use of PFPE materials such as magnetic resonance imaging, ultrasound imaging, x-ray fluoroscopy, Fourier transform infrared spectroscopy, ultraviolet or visible spectroscopy. PFPE materials are non ferromagnetic materials and, thus, are compatible with MRI. PFPE materials also have distinctive IR bands and have a very low optical density in the ultraviolet and visible wave lengths.

Parylene Underlayer for PFPE Materials

Parylene is a common name for a series of polymers based on paraxylene. The three most common types of parylene are: Parylene N, Parylene C, and Parylene D. The basis for the parylene family is the polyp-xylene monomer which comprises Parylene N. Parylene C and D are created by the substitution of a single chlorine molecule (C) or two (double) chlorine molecules (D). Parylene is an inert, hydrophobic, optically clear biocompatible polymer that is deposited via vapor deposition. Parylene can be coated onto virtually any type of surface and can be applied uniformly and “pin-hole free” in very thin coatings. The lubricity of parylene is similar to that of Teflon but can be applied uniformly in much thinner coatings than Teflon. As used herein, the term “parylene” shall include all types and variations of parylene, without limitation.

Applicants have unexpectedly found that parylene can serve as an excellent underlying layer for PFPE material coatings. For example, amino parylene provides an amino group that serves as an excellent anchor point for chemically attaching a PFPE layer thereto. A parylene underlayer can lower the surface energy of a device, thereby making the device smoother, and can also provide good electrical properties, while a PFPE coating provides protein absorption resistance. Parylene has an extremely high dielectric strength and, as such, is resistant to electrical breakdown which is important in implantable defibrillator devices. According to embodiments of the present invention, parylenes with various functional groups may be utilized including, but not limited to, amino groups which can act as anchors for complementary functional groups on PFPE materials.

Any of the above-described items including, but not limited to implantable intravascular electrophysiological devices, orthopedic apparatus configured to be implanted within the body of a subject, bandages configured to be applied to the body of a subject, surgical sutures, artificial blood vessels intraluminal prostheses, medical apparatus, implantable electronic devices, artificial tissue material for use within the lungs of a patient, material for use within a heart-lung machine, intraocular implants, contact lenses, cochlear implants, etc., may include a parylene underlayer and a coating of PFPE material.

Aneurysm Repair

PFPE materials may also be utilized in repairing blood vessel aneurysms. When a diseased blood vessel is exposed to hypertension, for example, dilation may occur at various localized regions. Typically these dilatations are produced at the region in the blood vessel wall which is weakest, whether inherently or as a result of disease or trauma. As the dilatation progresses, a more pronounced widening or sac, called an aneurysm, is produced, which may burst. According to embodiments of the present invention, a liquid PFPE material is injected into an aneurysm within a blood vessel and then cured as described above. The cured PFPE material serves as a filler that prevents the aneurysm from bursting.

Curing may include exposing the liquid PFPE material to light or other types of radiation, either directly or indirectly through, for example, the skin of the subject. Alternatively, curing may be facilitated by thermal energy from the body of a subject and/or from a source external to the subject. Curing may also be facilitated by providing curing initiators within the PFPE material, as described above. Alternatively, the liquid PFPE material may be provided as a two-component system that cures after the two components are mixed together. As described above, various additives can be added to PFPE materials according to embodiments of the present invention including, but not limited to, pharmacological agents, fillers, bioerodible materials, etc.

Colonic Diverticulosis Treatment

Colonic diverticulosis is a disease wherein mucosal and submucosal herniations develop through the circular muscle layer at weak points of the colonic wall. The sigmoid is the most commonly affected segment; however, diverticular disease also can involve the descending, ascending, and transverse colon as well as the jejunum, ileum, and duodenum.

According to embodiments of the present invention, a solution of PFPE material is injected into a herniation in the colon wall of a patient and then cured as described above, thereby sealing off the herniation and avoiding diverticulitis/rupture. Various ways of filling herniations with a PFPE solution, according to embodiments of the present invention, may be utilized. For example, a portion of a colon between two balloon segments is filled with a PFPE solution such that the PFPE solution fills the herniations. A colonoscope is then passed through a seal in the lower balloon and directs ultraviolet light, or other radiation, into each dirverticulum to cure the PFPE material. Preferably, the ultraviolet light, or other radiation, is shielded from exposure to the colon itself.

Curing may be facilitated by thermal energy from the body of a subject and/or from a source external to the subject. Curing may also be facilitated by providing curing initiators within the PFPE material, as described above. Alternatively, the liquid PFPE material may be provided as a two-component system that cures after the two components are mixed together. As described above, various additives can be added to PFPE materials according to embodiments of the present invention including, but not limited to, pharmacological agents, fillers, bioerodible materials, etc.

Photoinitiators

PFPE materials, according to embodiments of the present invention, and particularly for embodiments where PFPE materials are inserted within the body of a subject, beneath the skin of a subject, etc., may include photoinitiators to facilitate curing via light including, but not limited to, ultraviolet light, visible light and infrared light. Photoinitiators, which absorb light energy to form free radicals or other reactive intermediates, initiate polymerization. As known to those skilled in the art, Type I photoinitiators produce free radicals via intramolecular bond cleavage, e.g., arylalkyl ketones. As known to those skilled in the art, Type II photoinitiators produce free radicals via intermolecular electron transfer and hydrogen abstraction. Embodiments of the present invention may utilize any type of photoinitiator including Type I and Type II photoinitiators.

Photoinitiators that permit use of near infrared radiation (e.g., 650 to 900 nanometers) are particularly useful because biologic tissues, hemoglobin, water, and fat are least absorbent in this range. Exemplary photoinitiators that can be used in accordance with embodiments of the present invention and that can utilize near infrared radiation include, but are not limited to, photoinitiators for two-photon induced polymerizations such as 1-hydroxycyclohexyl phenyl ketone; 2,2-diethoxyacetophenone, benzophenone, Esacure TZT, blends of 4-methylbenzophenone and 2,4,6-trimethylbenzophenone; 4,4′-bis(diethylamino)benzophenone; 2-amino-5-nitrobenzophenone; isopropylthioxanthone; a mixture of the 2- and 4-isomers with triethanolamine as co-initiator; all of which are described in Near-IR Two-Photon Induced Polymerizations Using Either Benzophenone or Thioxanthone-Based Photoinitiators, by Brott et al., (Air Force Research Laboratory, 2001), which is incorporated herein by reference in its entirety. Another exemplary two-photon free-radical photopolymerization initiator that may be used in accordance with embodiments of the present invention is (E,E)₄-{2-[p-(N,N-di-n-butylamino)stilben-p-yl]vinyl}pyridine (abbreviated to DBASVP) (see, Synthesis, Structure and Properties of a New Two-Photon Photopolymerization Initiator, Ren et al., Journal of Materials Chemistry, 2002).

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. The invention is defined by the following claims, with equivalents of the claims to be included therein.

1. An implantable intravascular electrophysiological device, comprising:
a pulse generator configured for implantation within a blood vessel of a subject, wherein the pulse generator comprises a housing having a contour that allows blood flow through the vessel when implanted within the vessel, and wherein the pulse generator housing comprises a surface of PFPE material; and
one or more electrodes electrically coupled to the pulse generator, wherein the one or more electrodes are configured to be positioned within the blood vessel and to deliver electrical pulses thereto.

2. The intravascular electrophysiological device of claim 1, wherein the PFPE material includes one or more pharmacological agents configured to elute therefrom when the intravascular electrophysiological device is implanted within a blood vessel of a subject.

3. The intravascular electrophysiological device of claim 1, wherein the surface of PFPE material is smooth and continuous, and resists thrombus and endothelial formation thereon.

4. The intravascular electrophysiological device of claim 1, further comprising an anchor configured to secure the pulse generator housing within the blood vessel of the subject, wherein the anchor comprises a surface of PFPE material.

5. The intravascular electrophysiological device of claim 4, wherein the anchor PFPE material includes one or more pharmacological agents configured to elute therefrom when the anchor is implanted within a blood vessel of a subject.

6. The intravascular electrophysiological device of claim 1, further comprising a liner that is configured to be deployed within the vessel of a subject prior to implantation of the pulse generator housing, wherein the pulse generator housing is configured to be deployed within the liner after the liner has been deployed within the vessel, and wherein the liner comprises a surface of PFPE material.

7. The intravascular electrophysiological device of claim 6, wherein the liner has a length that is greater than a length of the pulse generator housing.

8. The intravascular electrophysiological device of claim 1, further comprising a removable implantation tool configured to facilitate implantation of the pulse generator housing within the blood vessel of the subject, wherein the removable implantation tool comprises PFPE material.

9. An implantable intravascular electrophysiological device, comprising:
a pulse generator configured for implantation within a blood vessel of a subject, wherein the pulse generator comprises a housing having a contour that allows blood flow through the vessel when implanted within the vessel, wherein the pulse generator housing comprises a surface of PFPE material, wherein the surface of PFPE material is smooth and continuous, and resists thrombus and endothelial formation thereon;
one or more electrodes electrically coupled to the pulse generator, wherein the one or more electrodes are configured to be positioned within the blood vessel and to deliver electrical pulses thereto; and
an anchor configured to secure the pulse generator housing within the blood vessel of the subject, wherein the anchor comprises a surface of PFPE material.

10. The intravascular electrophysiological device of claim 9, wherein the PFPE material includes one or more pharmacological agents configured to elute therefrom when the intravascular electrophysiological device is implanted within a blood vessel of a subject.

11. The intravascular electrophysiological device of claim 9, further comprising a liner that is configured to be deployed within the vessel of a subject prior to implantation of the pulse generator housing, wherein the pulse generator housing is configured to be deployed within the liner after the liner has been deployed within the vessel, and wherein the liner comprises a surface of PFPE material.

12. The intravascular electrophysiological device of claim 11, wherein the liner has a length that is greater than a length of the pulse generator housing.

13. The intravascular electrophysiological device of claim 9, further comprising a removable implantation tool configured to facilitate implantation of the pulse generator housing within the blood vessel of the subject, wherein the removable implantation tool comprises PFPE material.

14. An implantable intravascular electrophysiological device, comprising:
a pulse generator configured for implantation within a blood vessel of a subject, wherein the pulse generator comprises a housing having a contour that allows blood flow through the vessel when implanted within the vessel, and wherein the pulse generator housing comprises a surface of PFPE material;
one or more electrodes electrically coupled to the pulse generator, wherein the one or more electrodes are configured to be positioned within the blood vessel and to deliver electrical pulses thereto;
an anchor configured to secure the pulse generator housing within the blood vessel of the subject, wherein the anchor comprises a surface of PFPE material; and
a liner that is configured to be deployed within the vessel of a subject prior to implantation of the pulse generator housing, wherein the pulse generator housing is configured to be deployed within the liner after the liner has been deployed within the vessel, and wherein the liner comprises a surface of PFPE material.

15. The intravascular electrophysiological device of claim 14, wherein the PFPE material includes one or more pharmacological agents configured to elute therefrom when the intravascular electrophysiological device is implanted within a blood vessel of a subject.

16. The intravascular electrophysiological device of claim 14, wherein the pulse generator surface of PFPE material is smooth and continuous, and resists thrombus and endothelial formation thereon.

17. The intravascular electrophysiological device of claim 14, wherein the anchor surface of PFPE material is smooth and continuous, and resists thrombus and endothelial formation thereon.

18. The intravascular electrophysiological device of claim 14, wherein the liner surface of PFPE material is smooth and continuous, and resists thrombus and endothelial formation thereon.

19. The intravascular electrophysiological device of claim 14, wherein the liner has a length that is greater than a length of the pulse generator housing.

20. The intravascular electrophysiological device of claim 14, further comprising a removable implantation tool configured to facilitate implantation of the pulse generator housing within the blood vessel of the subject, wherein the removable implantation tool comprises a PFPE material.

21. An implantable intravascular electrophysiological device, comprising:
a pulse generator configured for implantation within a blood vessel of a subject, wherein the pulse generator comprises a housing having a contour that allows blood flow through the vessel when implanted within the vessel, and wherein the pulse generator housing comprises a surface of PFPE material overlying and attached to an underlying layer of parylene material;
one or more electrodes electrically coupled to the pulse generator, wherein the one or more electrodes are configured to be positioned within the blood vessel and to deliver electrical pulses thereto.

22. An orthopedic apparatus configured to be implanted within the body of a subject, wherein the apparatus comprises an outer surface of oxygen permeable, bacterial impermeable PFPE material attached to an underlying layer of parylene material.

23. A bandage configured to be applied to the body of a subject, wherein the bandage comprises oxygen permeable, bacterial impermeable PFPE material attached to an underlying layer of parylene material.

24. A surgical suture, comprising oxygen permeable, bacterial impermeable PFPE material attached to an underlying layer of parylene material, wherein the suture is configured to join tissue.

25. An artificial blood vessel for a subject, comprising oxygen permeable, bacterial impermeable PFPE material attached to an underlying layer of parylene material.

26. An intraluminal prosthesis having a tubular body portion comprising oxygen permeable, bacterial impermeable PFPE material attached to an underlying layer of parylene material.

27. A medical apparatus having a body portion, wherein the body portion comprises PFPE material attached to an underlying layer of parylene material.

28. An implantable electronic device, comprising:
a housing containing one or more electronic components therein; and
a PFPE material forming a hermetic seal around the housing that deters the ingress of moisture into the housing when the electronics device is implanted within the body of a subject, wherein the PFPE material is attached to an underlying layer of parylene material.

29. An artificial tissue material for use within the lungs of a patient, comprising a membrane of PFPE material that simulates alveolar action, wherein the PFPE material is attached to an underlying layer of parylene material.

30. A material for use within a heart-lung machine, comprising a membrane of PFPE material that enhances gas exchange during artificial respiration, wherein the PFPE material is attached to an underlying layer of parylene material.

31. An intraocular implant, comprising an oxygen permeable, bacterial impermeable liquid PFPE material attached to an underlying layer of parylene material.

32. A contact lens, comprising an oxygen permeable, bacterial impermeable liquid PFPE material attached to an underlying layer of parylene material.

33. A cochlear implant, comprising an oxygen permeable, bacterial impermeable liquid PFPE material attached to an underlying layer of parylene material.

34. A method of repairing a blood vessel aneurysm within the body of a subject, comprising:
injecting a liquid PFPE material into an aneurysm within a blood vessel; and
curing the liquid PFPE material such that the cured PFPE material serves as a filler that prevents the aneurysm from bursting.

35. The method of claim 34, wherein curing the liquid PFPE material comprises exposing the liquid PFPE material to light.

36. The method of claim 34, wherein curing the liquid PFPE material comprises exposing the liquid PFPE material to radiation.

37. The method of claim 34, wherein curing the liquid PFPE material is facilitated via thermal energy from the body of the subject.

38. The method of claim 34, wherein curing the liquid PFPE material is facilitated via thermal energy from a source external to the body of the subject.

39. The method of claim 34, wherein the liquid PFPE material comprises curing initiators.

40. A method of repairing a blood vessel aneurysm within the body of a subject, comprising:
injecting a liquid PFPE material into a blood vessel at the location of an aneurysm; and
curing the liquid PFPE material such that the cured PFPE material serves as a tubular prosthesis that elastically expands against the aneurysm and directs blood flow past the aneurysm.

41. The method of claim 40, wherein curing the liquid PFPE material comprises exposing the liquid PFPE material to light.

42. The method of claim 40, wherein curing the liquid PFPE material comprises exposing the liquid PFPE material to radiation.

43. The method of claim 40, wherein curing the liquid PFPE material is facilitated via thermal energy from the body of the subject.

44. The method of claim 40, wherein curing the liquid PFPE material is facilitated via thermal energy from a source external to the body of the subject.

45. The method of claim 40, wherein the liquid PFPE material comprises curing initiators.

46. A method of treating colonic diverticulosis, comprising:
filling a herniation in the colon wall of a subject with a liquid PFPE material; and
curing the PFPE material such that the cured PFPE material seals the herniation and prevents rupture thereof.

47. The method of claim 46, wherein curing the liquid PFPE material comprises exposing the liquid PFPE material to light.

48. The method of claim 46, wherein curing the liquid PFPE material comprises exposing the liquid PFPE material to light via a colonoscope.

49. The method of claim 46, wherein curing the liquid PFPE material comprises exposing the liquid PFPE material to radiation.

50. The method of claim 46, wherein curing the liquid PFPE material is facilitated via thermal energy from the body of the subject.

51. The method of claim 46, wherein curing the liquid PFPE material is facilitated via thermal energy from a source external to the body of the subject.

52. The method of claim 46, wherein the liquid PFPE material comprises curing initiators.

53. The method of claim 46, wherein the liquid PFPE material comprises one or more pharmacological agents.

54. The method of claim 46, wherein the liquid PFPE material comprises a two-component system that cures after the two components are mixed together.
